(19) United States
(12) Patent Application Publication
(10) Pub. No.: US 2017/0107559 A1
(43) Pub. Date: Apr. 20, 2017

(54) NUCLEIC ACID AMPLIFICATION REAGENT, NUCLEIC ACID AMPLIFICATION CARTRIDGE, AND NUCLEIC ACID AMPLIFICATION METHOD

(71) Applicant: Seiko Epson Corporation, Tokyo (JP)

(72) Inventors: Masayuki UEHARA, Matsumoto (JP); Kotaro IDEGAMI, Chino (JP)

(21) Appl. No.: 15/292,495

(22) Filed: Oct. 13, 2016

(30) Foreign Application Priority Data
Oct. 15, 2015 (JP) 2015-203475

(51) Int. Cl. C12Q 1/68; G01N 21/64

(52) U.S. Cl. CPC C12Q 1/686; G01N 21/6428; G01N 2021/6439

(57) ABSTRACT

A nucleic acid amplification reagent includes a first probe which anneals to a target nucleic acid in a region sandwiched between a site to which the 3′ end of a forward primer anneals and a site to which the 3′ end of a reverse primer anneals in a template nucleic acid, and a second probe which anneals to a nucleic acid other than the target nucleic acid in the region. Each of the first probe and the second probe includes a dye which emits light in a wavelength band mutually overlapping partially.

This application claims the benefit of Japanese Patent Application No. 2015-203475, filed on Oct. 15, 2015. The content of the aforementioned application is hereby incorporated by reference in its entirety.

### BACKGROUND

1. Technical Field

The present invention relates to a nucleic acid amplification reagent, a nucleic acid amplification cartridge, and a nucleic acid amplification method.

2. Related Art

A PCR (Polymerase Chain Reaction) method is a technique for amplifying a nucleic acid by repeating a cycle of temperature changes a plurality of times for the nucleic acid utilizing the occurrence of differences in denaturation and annealing of the nucleic acid due to a difference in the chain length of the nucleic acid or the like. By this technique, 2 to the nth power PCR products (n represents the number of cycles) are obtained.

As a nucleic acid amplification device using such a PCR method, a PCR device disclosed in JP-A-2012-115208 (PTL 1) has been proposed by the applicant of the invention. In a biochip mounted in the PCR device disclosed in PTL 1, a flow channel through which a reaction mixture containing a target nucleic acid and the like moves is formed, and the reaction mixture is placed in the flow channel, and also a liquid which has a lower specific gravity than that of the reaction mixture and is immiscible with the reaction mixture is filled.

In the PCR device disclosed in PTL 1, in the case where a biochip is mounted in a mounting section for mounting the biochip, a heating section which heats a first region of the flow channel formed in the biochip, and a heating section which heats a second region to a temperature which is different for the first region are included. Further, in the PCR device disclosed in PTL 1, a drive mechanism which changes the positions of the mounting section and the heating sections between a first position and a second position is included. By this drive mechanism, the reaction mixture in the biochip to be mounted in the mounting section reciprocally moves between the first region and the second region to be heated to different temperatures from each other. According to such a PCR device disclosed in PTL 1, the amplification reaction period can be reduced as compared with the case where the temperature of the entire biochip is changed to different temperatures from each other.

However, it was found that there is a tendency that light is not detected by a light detector even if a target nucleic acid is actually amplified under the conditions that the amplification reaction period is reduced using the PCR device disclosed in PTL 1 or the like.

### SUMMARY

An advantage of some aspects of the invention is to make a light detector easy to detect light.

An aspect of the invention is directed to a nucleic acid amplification reagent used for amplifying a target nucleic acid in a template nucleic acid, and including a first probe which anneals to the target nucleic acid in a region sandwiched between a site to which the 3′ end of a forward primer anneals and a site to which the 3′ end of a reverse primer anneals in the template nucleic acid, and a second probe which anneals to a nucleic acid other than the target nucleic acid in the region, wherein each of the first probe and the second probe includes a dye which emits light in a wavelength band mutually overlapping partially.

Another aspect of the invention is directed to a nucleic acid amplification cartridge including a liquid droplet containing the nucleic acid amplification reagent according to the aspect of the invention, and a container having a flow channel through which the liquid droplet moves.

Still another aspect of the invention is directed to a nucleic acid amplification method including a temperature setting step of setting the temperature of a first region of a container in which a liquid droplet containing the nucleic acid amplification reagent according to the aspect of the invention is placed to the denaturation temperature of a target nucleic acid, and also setting the temperature of a second region which is different from the first region to the synthesis temperature of the target nucleic acid, and an amplification step of repeating a cycle to undergo a denaturation stage in which the liquid droplet is moved to the first region and retained there and a synthesis stage in which the liquid droplet is moved to the second region and retained there a plurality of times.

In the nucleic acid amplification reagent according to the aspect of the invention, each of the first probe and the second probe includes a dye which emits light in a wavelength band mutually overlapping partially. Therefore, the intensity of light in a portion where the wavelength bands overlap with each other is higher than the intensity of light in a portion where the wavelength bands do not overlap with each other. That is, the target nucleic acid is labeled with the color developed by the dye of the first probe which anneals to the target nucleic acid, however, the developed color is enhanced by the color developed by the dye of the second probe. Therefore, the amount of the developed color for labeling the target nucleic acid is increased as compared with the case where the second probe is not used as in the case of the related art, and as a result, it becomes possible to make the light detector easy to detect light.

It is preferred that the second probe includes a probe which anneals to one strand in the region and a probe which anneals to the other strand in the region. In this case, the developed color for labeling the target nucleic acid present in one strand in the template nucleic acid is enhanced by both of the dye of the second probe which anneals to one strand thereof and the dye of the second probe which anneals to the other strand in the template nucleic acid. Therefore, the amount of the developed color for labeling the target nucleic acid is further increased as compared with the case where the second probe anneals only to either one strand or the other strand in the template nucleic acid.

From the viewpoint of increasing the amount of the developed color for labeling the target nucleic acid, it is preferred that the wavelength band of the light emitted by the dye included in the first probe and the wavelength band of the light emitted by the dye included in the second probe are entirely the same as compared with the case where these are partially the same. Further, it is preferred that the dye included in the first probe and the dye included in the second probe have the same chemical structure as compared with the case where these dyes have different chemical structures.

### BRIEF DESCRIPTION OF THE DRAWINGS

The invention will be described with reference to the accompanying drawings, wherein like numbers reference like elements.

FIG. 1 is a view showing a cross section of a nucleic acid amplification cartridge.

FIG. 2 is a view for illustrating primers and probes.

FIG. 3 is a view showing a state where a nucleic acid reagent solution is introduced into a container of a nucleic acid amplification cartridge.

FIG. 4 is a block diagram of a nucleic acid amplification device.

FIG. 5 is a view schematically showing a state of a rotation mechanism.

FIG. 6 is a view showing a state where a nucleic acid amplification cartridge is mounted in a mounting section.

FIG. 7A is a view showing a state of a thermal cycling treatment (A).

FIG. 7B is a view showing a state of a thermal cycling treatment (B).

FIG. 7C is a view showing a state of a thermal cycling treatment (C).

FIG. 7D is a view showing a state of a thermal cycling treatment (D).

FIG. 8 is a flowchart showing an amplification treatment procedure.

FIG. 9 is a graph showing amplification curves of Example 1 and Comparative Example 1.

### DESCRIPTION OF EXEMPLARY EMBODIMENTS

Hereinafter, embodiments for carrying out the invention will be described with reference to the accompanying drawings. The embodiments and the examples described below are provided only for the purpose of facilitating the understanding of the invention and not for the purpose of limiting the invention. The invention may be changed or modified without departing from the gist of the invention.

### (1) Embodiments

As embodiments, a nucleic acid amplification cartridge which is mountable in a nucleic acid amplification device for amplifying a target nucleic acid in a template nucleic acid will be described, and thereafter, the nucleic acid amplification device will be described.

### Nucleic Acid Amplification Cartridge

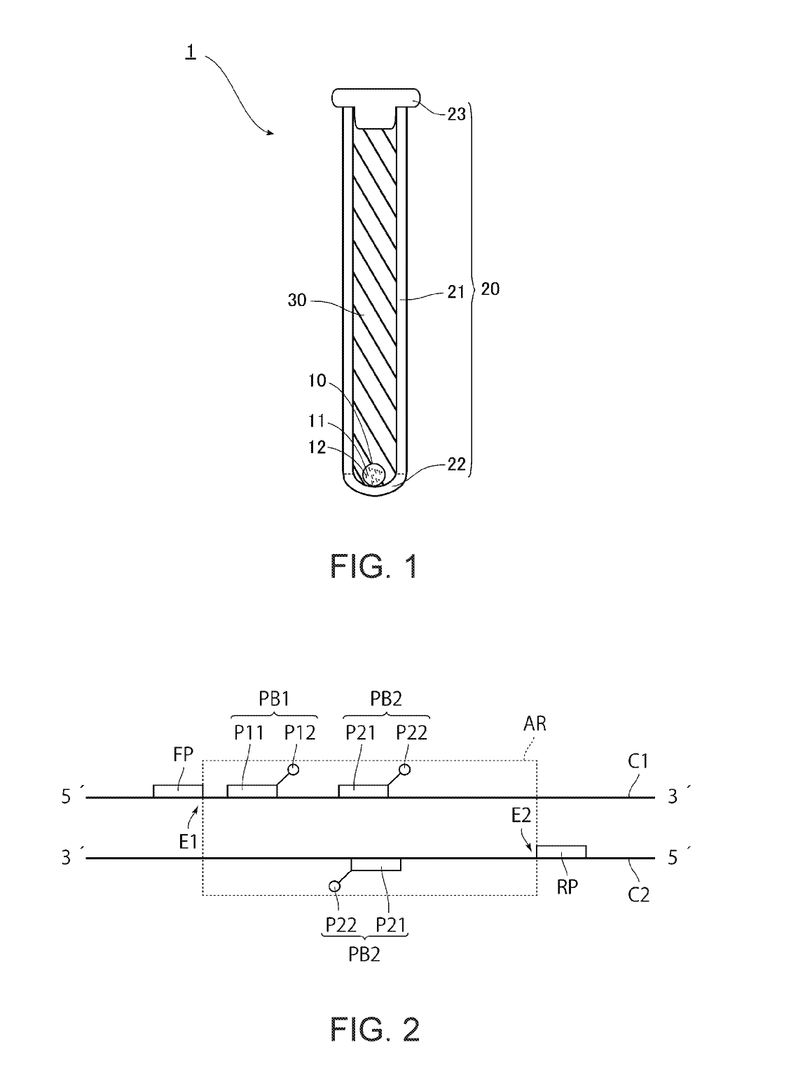

FIG. 1 is a view showing a cross section of a nucleic acid amplification cartridge 1. As shown in FIG. 1, the nucleic acid amplification cartridge 1 includes a liquid droplet 10 and a container 20.

The liquid droplet 10 is a place for allowing an amplification reaction of a nucleic acid to proceed, and contains a template nucleic acid 11 and a nucleic acid amplification reagent 12. In FIG. 1, the forms of the template nucleic acid 11 and the nucleic acid amplification reagent 12 are shown conveniently.

The template nucleic acid 11 is a double-stranded nucleic acid to be extracted from cells derived from a living organism such as a human or a bacterium, virus particles, or the like, and includes a target nucleic acid which is a nucleic acid fragment of an amplification target. Examples of the double-stranded nucleic acid include DNAs (deoxyribonucleic acids) and RNAs (ribonucleic acids).

The nucleic acid amplification reagent 12 is a reagent to be used for amplifying the target nucleic acid in the template nucleic acid 11. The nucleic acid amplification reagent 12 mainly includes a primer, a probe, a polymerase, and dNTPs (deoxyribonucleotide triphosphates). In the case where the template nucleic acid is a double-stranded RNA, in order to obtain a cDNA (complementary DNA) of the RNA, a reverse transcriptase, a primer for the reverse transcriptase, and the like are also contained in the nucleic acid amplification reagent 12.

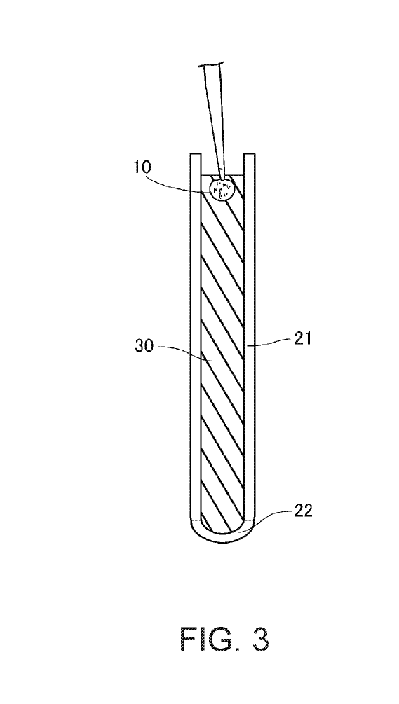

The primer is an oligonucleotide designed so as to anneal to the 3′ end or the 5′ end of the target nucleic acid, and as shown in FIG. 2, a forward primer FP which anneals to part of one strand C1 of a double strand in the template nucleic acid and a reverse primer RP which anneals to part of the other strand C2 of the double strand are included.

The probe is a target substance to be used for quantitatively determining the amount of an amplified nucleic acid, and in this embodiment, as shown in FIG. 2, a first probe PB1 and a second probe PB2 are included.

The first probe PB1 anneals to a target nucleic acid in a region AR sandwiched between a site to which the 3′ end E1 of the forward primer anneals and a site to which the 3′ end E2 of the reverse primer anneals. As the constituent elements of this first probe PB1, an annealing section P11 which anneals to the target nucleic acid and a dye P12 to be added to the annealing section P11 are included.

The annealing section P11 has a base sequence which anneals to the target nucleic acid. This base sequence may be a base sequence complementary to the entire base sequence of the target nucleic acid or may be a base sequence complementary to part of the base sequence of the target nucleic acid. Incidentally, the base sequence which specifically anneals to the target nucleic acid in the region AR is preferably used as the base sequence of the annealing section P11. As such a base sequence, for example, an artificial nucleic acid such as a PNA (peptide nucleic acid), an LNA (locked nucleic acid), or an ENA (ethylene bridged nucleic acid) is useful.

The dye P12 is a substance which emits light in a predetermined wavelength band. This dye P12 may be a fluorescent dye or a dye other than a fluorescent dye. Further, the dye P12 may be added to the end of the base sequence of the annealing section P11 or may be added to a site other than the end. In addition, the dye P12 may emit light in a state where it is added to the annealing section P11 or may emit light in a state where it is separated from the annealing section P11.

Examples of the dye which emits light in a state where it is separated from the annealing section P11 include a reporter dye and a quencher dye to be used for a TaqMan probe. The reporter dye and the quencher dye are added to the annealing section P11 such that the light emission of the reporter dye is suppressed by the quencher dye. When the reporter dye is separated from the annealing section P11 by an elongation reaction of a nucleic acid, the suppression of light emission by the quencher dye is released, and therefore, the reporter dye can emit light.

The second probe PB2 anneals to a nucleic acid other than the target nucleic acid in the region AR. In this embodiment, the second probe PB2 includes a probe which anneals to one strand C1 in the region AR and a probe which anneals to the other strand C2 in the region AR. As the constituent elements of this second probe PB2, an annealing section P21 which anneals to a nucleic acid other than the target nucleic acid in the region AR and a dye P22 to be added to the annealing section P21 are included.

The annealing section P21 has a base sequence which anneals to a first partial region other than the target nucleic acid in the region AR or a second partial region which is further part of the first partial region. This base sequence may be a base sequence complementary to the entire base sequence of the first partial region or the second partial region or may be a base sequence complementary to part of the base sequence of the first partial region or the second partial region.

The dye P22 is a substance which emits light in a predetermined wavelength band, and may be a fluorescent dye or a dye other than a fluorescent dye in the same manner as the dye P12. Further, the dye P22 may be added to the end of the base sequence of the annealing section P21 or may be added to a site other than the end in the same manner as the dye P12. In addition, the dye P22 may emit light in a state where it is added to the annealing section P21 or may emit light in a state where it is separated from the annealing section P21 in the same manner as the dye P12.

In this embodiment, the wavelength band of the light emitted by the dye P12 of the first probe PB1 and the wavelength band of the light emitted by the dye P22 of the second probe PB2 at least partially overlap with each other. That is, a portion where the wavelength bands are the same is present in the wavelength band of the light emitted by the dye P12 and in the wavelength band of the light emitted by the dye P22. Incidentally, it is preferred that the wavelength band of the light emitted by the dye P12 and the wavelength band of the light emitted by the dye P22 are entirely the same, however, they may be partially different from each other as long as they have a portion where the wavelength bands even slightly overlap with each other.

In the case where the wavelength band of the light emitted by the dye P12 and the wavelength band of the light emitted by the dye P22 are partially different from each other, it is preferred that an overlapping wavelength band between a wavelength band in a portion where the peak in the spectral distribution of the light emitted by the dye P12 becomes half and a wavelength band in a portion where the peak in the spectral distribution of the light emitted by the dye P22 becomes half is larger.

Further, it is preferred that the peak in the spectral distribution of the light emitted by the dye P12 and the peak in the spectral distribution of the light emitted by the dye P22 are closer to each other. In addition, in the case where each of the dye P12 and the dye P22 is a fluorescent dye, it is preferred that the molecular extinction coefficient and the fluorescence half-life of the dye P12 are closer to those of the dye P22. Further, the chemical structure of the dye P12 and the chemical structure of the dye P22 may be the same or different.

The polymerase is an enzyme which uses a single-stranded nucleic acid as a template and synthesizes a single strand which is a base sequence complementary to the nucleic acid used as the template, and the dNTPs are a mixture of four types of deoxyribonucleotide triphosphates (dATP, dCTP, dGTP, and dTTP).

As shown in FIG. 1, the container 20 includes a flow channel section 21 to serve as a flow channel through which the liquid droplet 10 can move, a bottom section 22 which closes an opening on one end side of the flow channel section 21, and a lid section 23 which closes an opening on the other end side of the flow channel section 21. In this embodiment, the flow channel section 21 is formed into, for example, a cylindrical shape, and the bottom section 22 is formed into, for example, a hollow hemispherical shape. Further, the lid section 23 is formed into, for example, a truncated conical shape, and is freely attachable to and detachable from the flow channel section 21.

In this container 20, an oil 30 is placed. The oil 30 has a lower specific gravity than that of a nucleic acid reagent solution to be introduced into the container 20 and is phase-separated from the nucleic acid reagent solution, and for example, 2CS silicone oil, a mineral oil, or the like is used.

The nucleic acid reagent solution is obtained, for example, as follows. That is, a specimen such as cells derived from a living organism such as a human or a bacterium or virus particles is collected with a collecting tool such as a cotton swab, and the template nucleic acid 11 is extracted from the specimen using a known extraction method. Subsequently, by using, for example, a solvent such as water (distilled water or sterile water) or a Tris-EDTA solution (TE), the nucleic acid reagent solution is prepared in a test tube or the like so that the concentrations of the template nucleic acid 11 and the respective components of the nucleic acid amplification reagent 12 are predetermined values. This nucleic acid reagent solution is introduced into the container 20 using a tool such as a pipette.

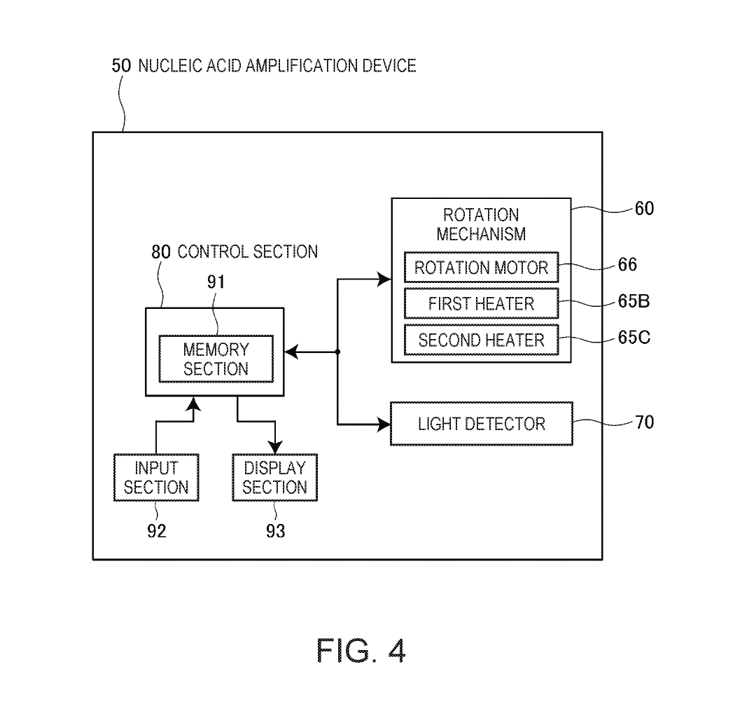

FIG. 3 is a view showing a state where the nucleic acid reagent solution is introduced into the container of the nucleic acid amplification cartridge. As shown in FIG. 3, in the case where the nucleic acid reagent solution is introduced into the container 20, since an action of making the surface area of the interface small acts on the nucleic acid reagent solution, the nucleic acid reagent solution is phase-separated from the oil 30 in the container 20 and therefore is transformed into the liquid droplet 10. The specific gravity of this liquid droplet 10 is higher than that of the oil 30, and therefore, the liquid droplet 10 sinks along the flow channel section 21.

### Nucleic Acid Amplification Device

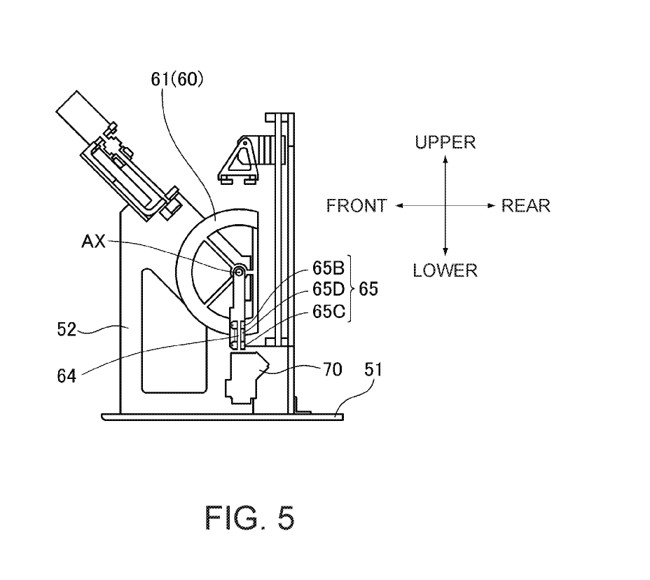

FIG. 4 is a block diagram of a nucleic acid amplification device. As shown in FIG. 4, a nucleic acid amplification device 50 includes a rotation mechanism 60, a light detector 70, and a control section 80.

### Rotation Mechanism

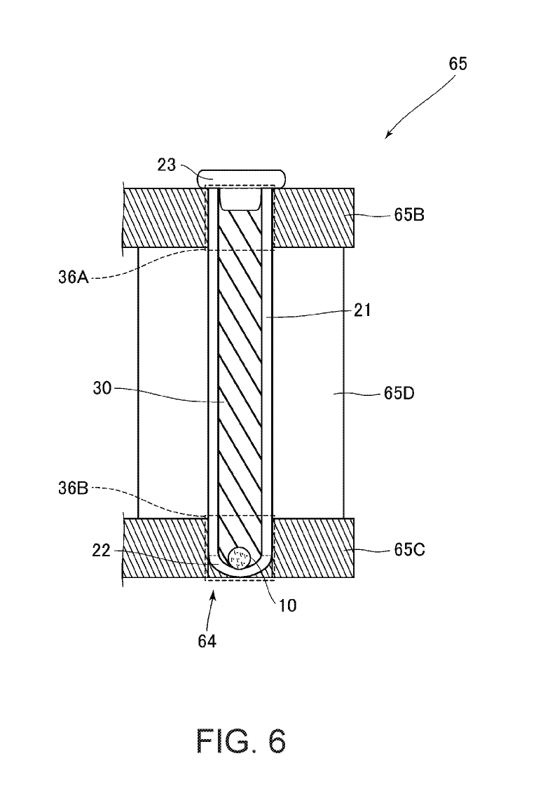

FIG. 5 is a view schematically showing a state of the rotation mechanism. FIG. 5 is a side view of the rotation mechanism 60. In the following description of the nucleic acid amplification device 50, as shown in FIG. 5, upper and lower, front and rear, and right and left are defined. That is, the vertical direction when a base 51 of the nucleic acid amplification device 50 is disposed horizontally is defined as “up and down direction”, and “upper” and “lower” are defined according to the direction of gravity. Further, the axis direction of the rotation axis AX of the nucleic acid amplification cartridge 1 is defined as “right and left direction”, and the direction perpendicular to the up and down direction and the right and left direction is defined as “front and rear direction”.

As shown in FIG. 5, the rotation mechanism 60 includes a rotating body 61 and a rotation motor 66 (FIG. 4) which rotates the rotating body 61. In the rotating body 61, a heater section 65 having an insertion hole 64 through which the nucleic acid amplification cartridge 1 can be put in and out is provided. The rotating body 61 rotates about the rotation axis AX supported by a support table 52 fixed to the base 51 without changing the relative position to the heater section 65 and the nucleic acid amplification cartridge 1 to be mounted in the insertion hole 64 of the heater section 65.

Incidentally, the insertion hole 64 of the heater section 65 in this embodiment functions as a hole through which the nucleic acid amplification cartridge 1 can be put in and out, and also functions as a mounting section for mounting the nucleic acid amplification cartridge 1 put in through the hole, however, the hole and the fitting section may be separately provided in the nucleic acid amplification device 50. Further, the number of mounting sections in which the nucleic acid amplification cartridge 1 can be mounted is not limited to 1, and may be 2 or more.

The rotation motor 66 (FIG. 4) rotates the rotating body 61 according to the instruction from the control section 80 such that the nucleic acid amplification cartridge 1 mounted in the insertion hole 64 of the heater section 65 turns upside down.

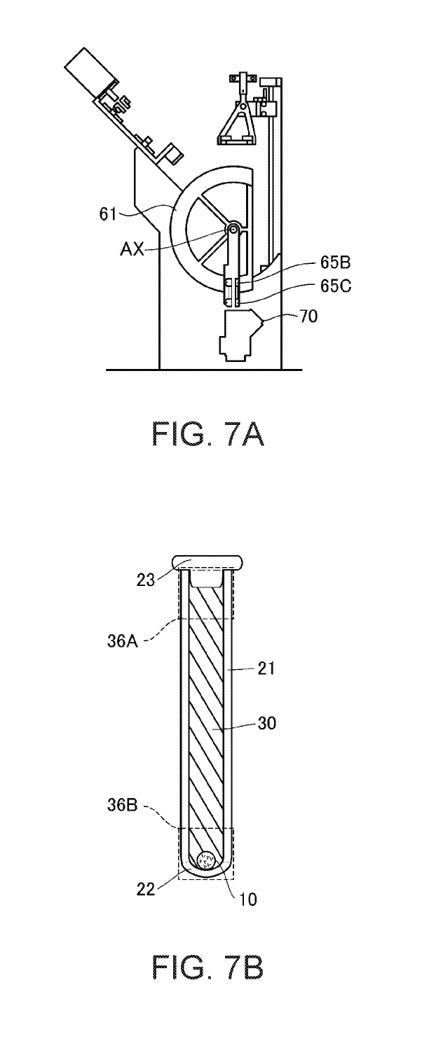

FIG. 6 is a view showing a state where the nucleic acid amplification cartridge is mounted in the mounting section. As shown in FIG. 6, the heater section 65 includes a first heater section 65B for heating a region to a temperature at which a denaturation reaction of the target nucleic acid proceeds and a second heater section 65C for heating a region to a temperature at which a synthesis reaction (an annealing reaction and an elongation reaction) of the target nucleic acid proceeds.

In the case where the nucleic acid amplification cartridge 1 is mounted in the insertion hole 64 of the heater section 65, a first region 36A located on the bottom section 22 side of the flow channel section 21 in the container 20 is surrounded by the first heater section 65B. The first heater section 65B heats the first region 36A to a preset temperature in the range of, for example, 95 to 100° C.

Further, in the case where the nucleic acid amplification cartridge 1 is mounted in the insertion hole 64 of the heater section 65, a second region 36B located on the lid section 23 side of the flow channel section 21 in the container 20 is surrounded by the second heater section 65C. The second heater section 65C heats the second region 36B to a preset temperature in the range of, for example, 50 to 75° C.

In this manner, the first region 36A of the container 20 in the nucleic acid amplification cartridge 1 is heated to a temperature at which the denaturation reaction of the target nucleic acid proceeds, and the second region 36B of the container 20 is heated to a temperature at which the synthesis reaction of the target nucleic acid proceeds.

Incidentally, between the first heater section 65B and the second heater section 65C, a spacer 65D which suppresses heat conduction between the first heater section 65B and the second heater section 65C is disposed. In this spacer 65D, a through-hole is formed at a position along the longitudinal direction of the insertion hole 64 of the first heater section 65B and the second heater section 65C, and the inhibition of insertion of the container 20 of the nucleic acid amplification cartridge 1 in the insertion hole 64 is prevented.

### Light Detector

The light detector 70 is a detector which detects the intensity of light emitted from the liquid droplet 10 placed in the container 20 of the nucleic acid amplification cartridge 1. As shown in FIG. 5, this light detector 70 is disposed, for example, in a state of facing the end of the nucleic acid amplification cartridge 1 mounted in the insertion hole 64 of the heater section 65 spaced apart at a predetermined distance.

The light detector 70 applies light corresponding to the dye P12 of the first probe PB1 and the dye P22 of the second probe PB2 according to the detection instruction from the control section 80 and detects the intensity of light emitted by the dyes P12 and P22. More specifically, the intensity of light in a portion where the wavelength band of the light emitted by the dye P12 of the first probe PB1 and the wavelength band of the light emitted by the dye P22 of the second probe PB2 overlap with each other is detected. For example, in the case where the dye P12 and the dye P22 are fluorescent dyes having the same chemical structure, excitation light corresponding to the fluorescent dyes is applied thereto, and the fluorescence intensity of the dye P12 and the dye P22 is detected.

Further, the light detector 70 provides data showing the light intensity obtained as the detection result to the control section 80. The light intensity shown by the data reflects the number of times of occurrence of the synthesis reaction (annealing reaction and elongation reaction) of the target nucleic acid. Therefore, it is shown that as the light intensity shown by the data provided to the control section 80 is higher, the number of target nucleic acids (the number of amplified copies) is larger.

### Control Section

As shown in FIG. 4, the control section 80 includes a memory section 91, and to the control section 80, an input section 92, a display section 93, and the like are connected. In the memory section 91, a region in which a program is stored, a region in which a variety of data such as setting data to be input from the input section 92 and data obtained by a nucleic acid amplification treatment are stored, and a region in which the program and the data are expanded are included.

The control section 80 appropriately controls the rotation mechanism 60 and the light detector 70 based on the program and the setting data stored in the memory section 91, and a thermal cycling treatment or an amplification analysis treatment is appropriately performed.

### Thermal Cycling Treatment

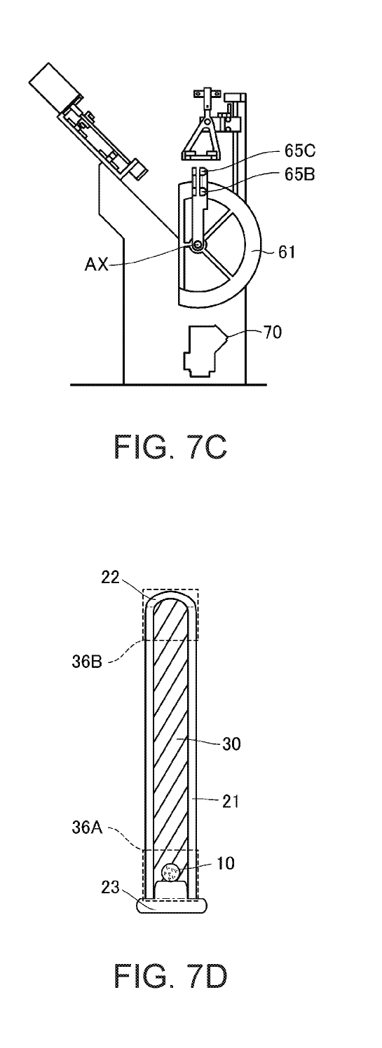

FIGS. 7A to 7D are views showing a state of a thermal cycling treatment. More specifically, FIGS. 7A and 7B show a state of a synthesis stage of a target nucleic acid, and FIGS. 7C and 7D show a state of a denaturation stage of the target nucleic acid.

That is, for example, when receiving a command to perform a thermal cycling treatment from the input section 92, the control section 80 drives the first heater section 65B provided in the rotating body 61, and heats the first region 36A of the container 20 in the nucleic acid amplification cartridge 1 to a temperature at which the denaturation reaction of the target nucleic acid proceeds. Further, the control section 80 drives the second heater section 65C provided in the rotating body 61, and heats the second region 36B of the container 20 in the nucleic acid amplification cartridge 1 to a temperature at which the synthesis reaction of the target nucleic acid proceeds. By doing this, a temperature gradient is formed in the oil 30 filled in the container 20 of the nucleic acid amplification cartridge 1.

It takes a predetermined period from when the first heater section 65B and the second heater section 65C are driven to when the temperature of the oil 30 in the first region 36A has reached, for example, 98° C. and the temperature of the oil 30 in the second region 36B has reached, for example, 54° C. During this period, the amplification reaction of the target nucleic acid does not properly proceed, and therefore, the control section 80 waits during this period as a waiting period.

At this time, as shown in FIGS. 7A and 7B, the rotating body 61 is positioned at a standard position where a portion on the lid section 23 side of the container 20 mounted in the insertion hole 64 of the heater section 65 is disposed on the upper side, and a portion on the bottom section 22 side of the container 20 is disposed on the lower side. In the case where the rotating body 61 is positioned at the standard position, the liquid droplet 10 sinks in the flow channel section 21 by its own weight and is retained in the second region 36B. Therefore, the target nucleic acid contained in the liquid droplet 10 is not transferred to the first round of the denaturation stage.

When the above-mentioned waiting period has elapsed, the control section 80 rotates the rotating body 61 by 180 degrees. In this case, as shown in FIGS. 7C and 7D, the rotating body 61 is positioned at an inverted position where a portion on the lid section 23 side of the container 20 mounted in the insertion hole 64 of the heater section 65 is disposed on the lower side, and a portion on the bottom section 22 side of the container 20 is disposed on the upper side. In the case where the rotating body 61 is positioned at the inverted position, the liquid droplet 10 sinks in the flow channel section 21 by its own weight and moves to the first region 36A. Therefore, the target nucleic acid contained in the liquid droplet 10 is transferred to the denaturation stage.

Further, the control section 80 stops the rotating body 61 only in a denaturation reaction period set as a period of the denaturation stage of the target nucleic acid from when the rotation of the rotating body 61 by 180 degrees is completed (the rotating body 61 is stopped). By doing this, the denaturation reaction of the target nucleic acid contained in the liquid droplet 10 proceeds. Incidentally, the denaturation reaction period is set to at least a period equal to or more than a period in which the liquid droplet 10 moves between the first region 36A and the second region 36B through the flow channel section 21. More specifically, a period of 5 seconds or more and less than 30 seconds is adopted as a general denaturation reaction period, however, a period of 2 seconds or more and less than 5 seconds which is shorter than the general denaturation reaction period may be adopted as the denaturation reaction period.

Subsequently, when the denaturation reaction period has elapsed, the control section 80 changes the position of the rotating body 61 from the inverted position to the standard position by rotating the rotating body 61 by 180 degrees, and as shown in FIG. 7B, the liquid droplet 10 is moved to the second region 36B. By doing this, the target nucleic acid contained in the liquid droplet 10 is transferred to the synthesis stage.

Further, the control section 80 stops the rotating body 61 only in a synthesis reaction period set as a period of the synthesis stage of the target nucleic acid from when the rotation of the rotating body 61 by 180 degrees is completed (the rotating body 61 is stopped). By doing this, the annealing reaction and the elongation reaction of the target nucleic acid contained in the liquid droplet 10 proceed. Incidentally, the synthesis reaction period is set to at least a period equal to or more than a period in which the liquid droplet 10 moves between the first region 36A and the second region 36B through the flow channel section 21 in the same manner as the above-mentioned denaturation reaction period. More specifically, a period of 20 seconds or more and less than 60 seconds is adopted as a general synthesis reaction period, however, a period of 3 seconds or more and less than 20 seconds which is shorter than the general synthesis reaction period may be adopted as the synthesis reaction period.

In this manner, the control section 80 repeats a cycle to undergo the denaturation stage in which the liquid droplet 10 is moved to the first region 36A and retained there and the synthesis stage in which the liquid droplet 10 is moved to the second region 36B and retained there a plurality of times by alternately changing the position between the inverted position and the standard position. The number of cycles to be repeated is set in the control section 80, and for example, set to 50.

### Amplification Analysis Treatment

The amplification analysis treatment is performed in parallel with the thermal cycling treatment at the same time. That is, the control section 80 gives a detection instruction to the light detector 70 for each synthesis reaction period, and stores data showing the light intensity provided from the light detector 70 as the result of the detection instruction in the memory section 91.

As shown in FIGS. 7A and 7B, in the synthesis reaction period, the rotating body 61 is positioned at the standard position, and therefore, the liquid droplet 10 in the container 20 sinks toward the bottom section 22. However, immediately after the rotating body 61 is positioned at the standard position, the liquid droplet 10 has not yet reached the bottom section 22 in some cases. Therefore, the time when the control section 80 gives a detection instruction to the light detector 70 is desirably after a predetermined time has elapsed from when the rotation of the rotating body 61 from the inverted position to the standard position is completed. In particular, it is desirably immediately before the rotating body 61 is rotated from the standard position to the inverted position.

When receiving data showing the light intensity obtained for the number of times which is set as the number of cycles to be repeated, the control section 80 creates an amplification curve showing a change in the light intensity with respect to the number of cycles based on the data obtained for the number of times. When creating the amplification curve, the control section 80 determines acceptance or rejection with respect to the reference amplification efficiency based on the amplification curve, and appropriately causes the display section 93 to display both or either of the determination result and the amplification curve.

### Amplification Treatment Procedure

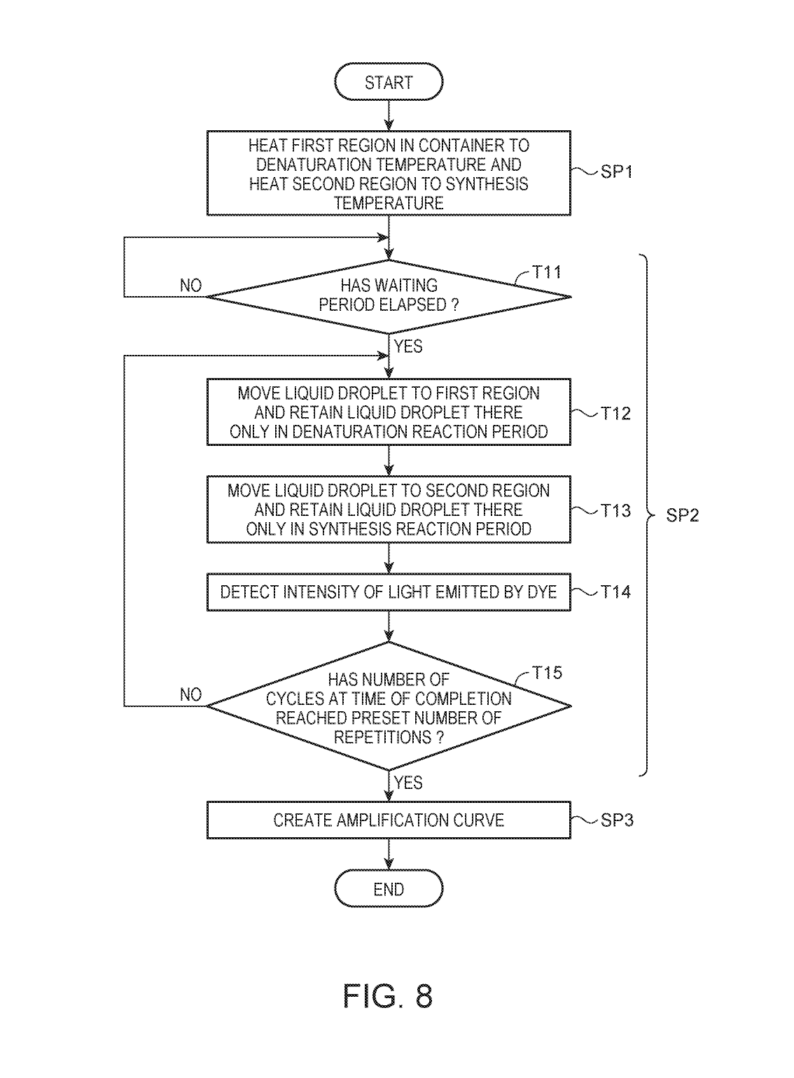

Next, an amplification treatment procedure which is a series of the above-mentioned respective treatments performed by the control section 80 will be described. FIG. 8 is a flowchart showing the amplification treatment procedure. As shown in FIG. 8, the control section 80 proceeds to a heating step SP1, and heats the first region 36A of the container 20 in the nucleic acid amplification cartridge 1 to the denaturation temperature set as a temperature at which the denaturation reaction of the target nucleic acid proceeds. Further, the control section 80 heats the second region 36B of the container 20 to the synthesis temperature set as a temperature at which the synthesis reaction of the target nucleic acid proceeds, and then, proceeds to an amplification step SP2.

In a first stage T11 of the amplification step SP2, the control section 80 waits until a waiting period set as a period in which the temperature of a heating target has reached a desired temperature from when heating is started has elapsed, and when the waiting period has elapsed, the control section 80 proceeds to a second stage T12 of the amplification step SP2.

In the second stage T12 of the amplification step SP2, the control section 80 rotates the rotating body 61 from the standard position to the inverted position so that the liquid droplet 10 is moved to the denaturation temperature region (first region 36A) of the container 20. Subsequently, the control section 80 keeps the rotating body 61 stopping so that the liquid droplet 10 is retained in the denaturation temperature region of the container 20 until the denaturation reaction period has elapsed from when the rotating body 61 is positioned at the inverted position. When the denaturation reaction period has elapsed, the control section 80 proceeds to a third stage T13 of the amplification step SP2.

In the third stage T13 of the amplification step SP2, the control section 80 rotates the rotating body 61 from the inverted position to the standard position so that the liquid droplet 10 is moved to the synthesis temperature region (second region 36B) of the container 20. Subsequently, the control section 80 keeps the rotating body 61 stopping so that the liquid droplet 10 is retained in the synthesis temperature region of the container 20 until the synthesis reaction period has elapsed from when the rotating body 61 is positioned at the standard position. When the synthesis reaction period has elapsed, the control section 80 proceeds to a fourth stage T14 of the amplification step SP2.

In the fourth stage T14 of the amplification step SP2, the control section 80 causes the light detector 70 to detect the intensity of light in a portion where the wavelength band of the light emitted by the dye P12 of the first probe PB1 and the wavelength band of the light emitted by the dye P22 of the second probe PB2 overlap with each other. Further, when receiving data showing the light intensity as the detection result from the light detector 70, the control section 80 proceeds to a fifth stage T15 of the amplification step SP2.

In the fifth stage T15 of the amplification step SP2, the control section 80 recognizes whether the number of cycles at the time of completion has reached the number of repetitions set as the number of cycles to be repeated. Here, when the number of cycles at the time of completion has not reached the preset number of repetitions, the control section 80 increases the number of cycles at the time of completion by one, and thereafter returns to the first stage T11 of the amplification step SP2 and repeats the above-mentioned treatments. On the other hand, when the number of cycles at the time of completion has reached the preset number of repetitions, the control section 80 proceeds to an amplification curve creation step SP3.

In the amplification curve creation step SP3, the control section 80 creates an amplification curve using data showing the intensity of light for each number of cycles to be repeated, and the heating of the first region 36A and the second region 36B in the container 20 is stopped. Thereafter, the control section 80 completes the amplification treatment procedure.

### Outline

As described above, the nucleic acid amplification reagent 12 of this embodiment includes a first probe PB1 and a second probe PB2. The first probe PM anneals to a target nucleic acid in a region AR sandwiched between a site to which the 3′ end E1 of a forward primer FP anneals and a site to which the 3′ end E2 of a reverse primer RP anneals in a template nucleic acid. On the other hand, the second probe PB2 anneals to a nucleic acid other than the target nucleic acid in the region AR.

The first probe PM and the second probe PB2 have a dye P12 and a dye P22, respectively, each of which emits light in a wavelength band mutually overlapping partially. Due to this, the intensity of light in a portion where the wavelength bands overlap with each other is higher than the intensity of light in a portion where the wavelength bands do not overlap with each other. That is, the target nucleic acid is labeled with the color developed by the dye P12, however, the developed color is enhanced by the color developed by the dye P22.

Therefore, according to the nucleic acid amplification reagent 12 of this embodiment, the amount of the developed color for labeling the target nucleic acid is increased as compared with the case where the second probe PB2 is not used as in the case of the related art, and as a result, it becomes possible to make the light detector 70 easy to detect light.

Further, in this embodiment, the second probe PB2 includes a probe which anneals to one strand C1 in the region AR and a probe which anneals to the other strand C2 in the region AR.

Due to this, the developed color for labeling the target nucleic acid present in one strand C1 in the template nucleic acid is enhanced by both of the dye P22 of the second probe PB2 which anneals to one strand C1 thereof and the dye P22 of the second probe PB2 which anneals to the other strand C2 in the template nucleic acid. Therefore, the amount of the developed color for labeling the target nucleic acid is further increased as compared with the case where the second probe PB2 anneals only to either one strand C1 or the other strand C2 in the template nucleic acid, and as a result, it becomes possible to make the light detector 70 more easier to detect light.

From the viewpoint of increasing the amount of the developed color for labeling the target nucleic acid, it is preferred that the wavelength band of the light emitted by the dye P12 included in the first probe PM and the wavelength band of the light emitted by the dye P22 included in the second probe PB2 are entirely the same as compared with the case where these are partially the same. Further, it is preferred that the dye P12 included in the first probe PM and the dye P22 included in the second probe PB2 have the same chemical structure as compared with the case where these dyes have different chemical structures.

In the case where the amplification reaction period (the denaturation reaction period and the synthesis reaction period) is reduced to less than 25 seconds, which is a generally adopted period, there is a tendency that light is not detected by the light detector 70 even if the target nucleic acid is actually amplified. Therefore, in the case where the amplification reaction period is reduced, the nucleic acid amplification reagent 12 of this embodiment is particularly useful.

### (2) Modification Examples

In the above embodiment, the second probe PB2 includes a probe which anneals to one strand C1 in the region AR and a probe which anneals to the other strand C2 in the region AR. However, either one of the probes may be omitted. However, from the viewpoint of increasing the amount of the developed color for labeling the target nucleic acid, as described above, it is preferred that the second probe PB2 includes a probe which anneals to one strand C1 in the region AR and a probe which anneals to the other strand C2 in the region AR.

Further, in the above embodiment, the number of dyes P12 included in the first probe PB1 and the number of dyes P22 included in the second probe PB2 are both set to 1, however, the number of dyes may be 2 or more. Further, the number of dyes P12 included in the first probe PB1 may be different from the number of dyes P22 included in the second probe PB2.

Further, in the above embodiment, the amplification reaction is performed in a shorter period than a general denaturation reaction period and synthesis reaction period, however, the amplification reaction may be performed in the general denaturation reaction period and synthesis reaction period.

Further, in the above embodiment, the specific gravity of the liquid droplet 10 is higher than the specific gravity of the oil 30. However, the specific gravity of the liquid droplet 10 may be lower than the specific gravity of the oil 30. Also in this case, the same advantageous effects as those of the above embodiment are obtained.

Further, in the above embodiment, the start time of the denaturation reaction period and the synthesis reaction period is set to the time when the rotation of the rotating body 61 by 180 degrees is completed (the rotating body 61 is stopped), however, the start time may be set to the time when the rotation of the rotating body 61 by 180 degrees starts.

Further, in the above embodiment, the rotation mechanism 60 is adopted as a mechanism for alternately moving the liquid droplet 10 in the container 20 in the nucleic acid amplification cartridge 1 between the first region 36A and the second region 36B. However, any of various drive mechanisms other than the above rotation mechanism 60 can be applied as long as it is a drive mechanism for alternately moving the liquid droplet 10 between the first region 36A which is brought to the denaturation temperature of the target nucleic acid and the second region 36B which is a different region from the first region 36A and is brought to the synthesis temperature of the target nucleic acid in the container 20. Further, a common nucleic acid amplification device which does not have a drive mechanism for alternately moving the liquid droplet 10 may be applied.

Further, in the above embodiment, as the region to which the liquid droplet 10 should be moved in the container 20, the first region 36A which is brought to the denaturation temperature of the target nucleic acid, and the second region 36B which is a different region from the first region 36A and is brought to the synthesis temperature of the target nucleic acid are disposed. However, three regions may be disposed. That is, as the first region 36A, a region which is brought to the denaturation temperature of the target nucleic acid is disposed. Further, as the second region 36B, two regions which are different from each other are disposed, and one region is brought to the annealing temperature set as a temperature at which the annealing reaction in the synthesis reaction of the target nucleic acid proceeds, and the other region is brought to the elongation temperature set as a temperature at which the elongation reaction of the target nucleic acid proceeds. In this manner, the invention is not limited to the above embodiment, in which the temperature changes in one cycle in the following two stages: a denaturation stage and a synthesis stage, and even in the case where the temperature changes in one cycle in the following three stages: a denaturation stage, an annealing stage, and an elongation stage, the liquid droplet 10 can be moved in the container. Incidentally, even in the case where the temperature changes in one cycle in three stages, any of various moving mechanisms other than the rotation mechanism can be applied.

In the above embodiment, the nucleic acid amplification device 50 including the first heater section 65B and the second heater section 65C is applied. However, a nucleic acid amplification device other than the nucleic acid amplification device 50 of the above embodiment may be applied as long as a temperature gradient can be formed inside the container 20.

### EXAMPLES

### Example 1

First, a template nucleic acid 11, a nucleic acid amplification reagent 12, and a buffer were added to a test tube, and thereafter, distilled water was added thereto, whereby 10 μL of a nucleic acid reagent solution was prepared.

As the template nucleic acid 11, 250 copies of genomic DNA of Mycoplasma pneumoniae manufactured by Vircell were used and added to the test tube in an amount of 0.625 μL.

As the polymerase to be contained in the nucleic acid amplification reagent 12, PlatinumTaq manufactured by Life Technologies Corporation was used and added to the test tube in an amount of 0.4 μL.

As the dNTPs to be contained in the nucleic acid amplification reagent 12, dNTPs manufactured by Roche were used and added to the test tube in an amount of 0.5 μL.

As the forward primer and the reverse primer to be contained in the nucleic acid amplification reagent 12, a forward primer and a reverse primer were added to the test tube in an amount of 0.8 μL each.

As the first probe PB1 to be contained in the nucleic acid amplification reagent 12, a TaqMan probe (fluorescent probe) was used and added to the test tube in an amount of 0.6 μL.

As the second probe PB2 to be contained in the nucleic acid amplification reagent 12, a TaqMan probe (fluorescent probe) was used and added to the test tube in an amount of 0.6 μL.

As the buffer, a buffer with a composition of 25 mM MgCl₂, 250 mM Tris-HCl (pH 9.0), and 125 mM KCl was used and added to the test tube in an amount of 2.0 μL.

The sequences of the forward primer, the reverse primer, the first probe PB1, and the second probe PB2 are shown in the following Table 1. Incidentally, the dye P12 of the first probe PB1 and the dye P22 of the second probe PB2 have the same chemical structure.

TABLE 1

| | |
|---|---|
| Forward | 5′-TCGGTGAAATCCAGGTACGGGT-3′ |
| prime | |
| Reverse | 5′-GCGAACTTGCATCGATTGCTCC-3′ |
| prime | |
| First | 5′-FAM-TTGCGCCTAACGGGTGTCTT-BHQ-3′ |
| probe | |
| Second | 5′-FAM-TCTCTACATGATAATGTCCTGATCA-BHQ-3′ |
| probe | |

“FAM” in the above Table 1 is the abbreviation of “fluorescein aminohexyl” and is one of the reporter fluorescent dyes. Further, “BHQ” in the above Table 1 is the abbreviation of “Black Hole Quencher” and is one of the quencher fluorescent dyes.

Subsequently, 1.6 μL of the nucleic acid reagent solution was introduced into the container 20, and the liquid droplet 10 was formed in the container 20, and thereafter, the container 20 was mounted in the insertion hole 64 of the heater section 65 in the nucleic acid amplification device 50, and then, the thermal cycling treatment and the amplification analysis treatment were performed.

The number of cycles in the thermal cycling treatment was set to 50, the denaturation reaction period was set to 4 seconds, the synthesis reaction period was set to 6 seconds, the temperature of the first heater section 65B was set to 98° C., and the temperature of the second heater section 65C was set to 54° C.

### Comparative Example 1

A nucleic acid reagent solution was prepared under the same conditions as those of Example 1 described above without using the second probe PB2 in Example 1.

Subsequently, the nucleic acid reagent solution was introduced into the container 20, and the liquid droplet 10 was formed in the container 20, and thereafter, the container 20 was mounted in the insertion hole 64 of the heater section 65 in the nucleic acid amplification device 50, and then, the thermal cycling treatment and the amplification analysis treatment were performed under the same conditions as those of Example 1 described above.

### Comparison Between Example 1 and Comparative Example 1

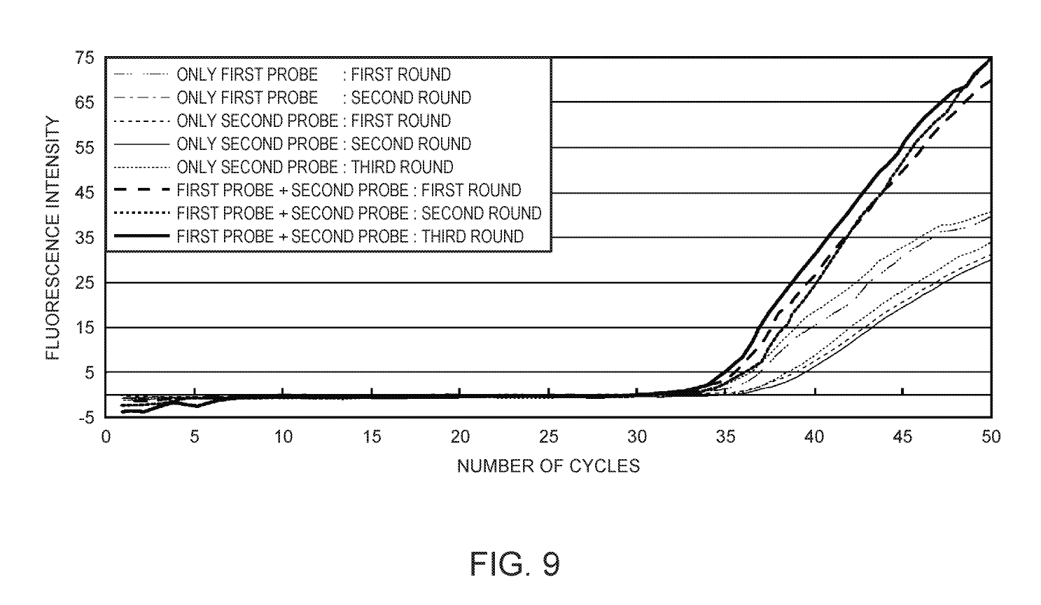

The amplification curves for Example 1 and Comparative Example 1 described above are shown in FIG. 9. As shown in FIG. 9, it was found that in the case where the second probe PB2 was used, the amount of change in the fluorescence intensity is significantly improved even when the denaturation reaction period and the synthesis reaction period were reduced from the generally adopted period as compared with the case where the second probe PB2 was not used.

What is claimed is:

1. A nucleic acid amplification reagent, which is used for amplifying a target nucleic acid in a template nucleic acid, comprising:
a first probe which anneals to the target nucleic acid in a region sandwiched between a site to which the 3′ end of a forward primer anneals and a site to which the 3′ end of a reverse primer anneals in the template nucleic acid; and
a second probe which anneals to a nucleic acid other than the target nucleic acid in the region, wherein
each of the first probe and the second probe includes a dye which emits light in a wavelength band mutually overlapping partially.

2. The nucleic acid amplification reagent according to claim 1, wherein the second probe includes a probe which anneals to one strand in the region and a probe which anneals to the other strand in the region.

3. The nucleic acid amplification reagent according to claim 1, wherein the wavelength band of the light emitted by the dye included in the first probe and the wavelength band of the light emitted by the dye included in the second probe are the same.

4. The nucleic acid amplification reagent according to claim 1, wherein the dye included in the first probe and the dye included in the second probe have the same chemical structure.

5. The nucleic acid amplification reagent according to claim 1, wherein the reagent includes the forward primer and the reverse primer.

6. The nucleic acid amplification reagent according to claim 1, wherein the reagent includes a polymerase and dNTPs.

7. A nucleic acid amplification cartridge, comprising:
a liquid droplet containing a template nucleic acid and the nucleic acid amplification reagent according to claim 1; and
a container having a flow channel through which the liquid droplet moves.

8. A nucleic acid amplification cartridge, comprising:
a liquid droplet containing a template nucleic acid and the nucleic acid amplification reagent according to claim 2; and
a container having a flow channel through which the liquid droplet moves.

9. A nucleic acid amplification cartridge, comprising:
a liquid droplet containing a template nucleic acid and the nucleic acid amplification reagent according to claim 3; and
a container having a flow channel through which the liquid droplet moves.

10. A nucleic acid amplification cartridge, comprising:
a liquid droplet containing a template nucleic acid and the nucleic acid amplification reagent according to claim 4; and
a container having a flow channel through which the liquid droplet moves.

11. A nucleic acid amplification cartridge, comprising:
a liquid droplet containing a template nucleic acid and the nucleic acid amplification reagent according to claim 5; and
a container having a flow channel through which the liquid droplet moves.

12. A nucleic acid amplification cartridge, comprising:
a liquid droplet containing a template nucleic acid and the nucleic acid amplification reagent according to claim 6; and
a container having a flow channel through which the liquid droplet moves.

13. A nucleic acid amplification method, comprising:
a heating step of heating a first region of a container in which a liquid droplet containing a template nucleic acid and the nucleic acid amplification reagent according to claim 1 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.

14. A nucleic acid amplification method, comprising:
a heating step of heating a first region of a container in which a liquid droplet containing a template nucleic acid and the nucleic acid amplification reagent according to claim 2 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.

15. A nucleic acid amplification method, comprising:
a heating step of heating a first region of a container in which a liquid droplet containing a template nucleic acid and the nucleic acid amplification reagent according to claim 3 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.

16. A nucleic acid amplification method, comprising:
a heating step of heating a first region of a container in which a liquid droplet containing a template nucleic acid and the nucleic acid amplification reagent according to claim 4 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.

17. A nucleic acid amplification method, comprising:
a heating step of heating a first region of a container in which a liquid droplet containing a template nucleic acid and the nucleic acid amplification reagent according to claim 5 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.

18. A nucleic acid amplification method, comprising:
a heating step of heating a first region of a container in which a liquid droplet containing a template nucleic acid and the nucleic acid amplification reagent according to claim 6 is placed to the denaturation temperature of the target nucleic acid, and also heating a second region which is different from the first region to the synthesis temperature of the target nucleic acid; and
an amplification step of repeating a cycle to undergo a denaturation stage in which the liquid droplet is moved to the first region and retained there and a synthesis stage in which the liquid droplet is moved to the second region and retained there a plurality of times.


SEQUENCE LISTING (4 sequences)

SEQ ID NO: 1 - DNA, 22 bp, Artificial Sequence
  forward primer
tcggtgaaatccaggtacgggt

SEQ ID NO: 2 - DNA, 22 bp, Artificial Sequence
  reverse primer
gcgaacttgcatcgattgctcc

SEQ ID NO: 3 - DNA, 20 bp, Artificial Sequence
  first probe
ttgcgcctaacgggtgtctt

SEQ ID NO: 4 - DNA, 25 bp, Artificial Sequence
  second probe
tctctacatgataatgtcctgatca